(19) United States
(12) Patent Application Publication
(10) Pub. No.: US 2023/0248239 A1
(43) Pub. Date: Aug. 10, 2023

(54) MULTI-FUNCTIONAL TELEMEDICAL DEVICE

(71) Applicant: Michael Francis Mersinger, Clarendon Hills, IL (US)

(72) Inventor: Michael Francis Mersinger, Clarendon Hills, IL (US)

(21) Appl. No.: 18/004,995

(22) PCT Filed: Jul. 23, 2021
(86) PCT No.: PCT/US21/42962

§ 371 Date: Jan. 10, 2023

Related U.S. Application Data
(60) Provisional application No. 63/055,681, filed on Jul. 23, 2020.

(51) Int. Cl. A61B 5/00; A61B 5/1455; A61B 5/024; A61B 5/022

(52) U.S. Cl. CPC A61B 5/0022; A61B 5/14551; A61B 5/024; A61B 5/022; A61B 5/742

(57) ABSTRACT

A multi-functional telemedical device that includes a housing configured for hand-held manipulation having an end that is configured to be fully rotational about an axis; a camera disposed on a front side of the end and communicatively; multi-functional viewing tool disposed a second side of the rotatable end and opposite the camera; an auscultation sensor communicatively; a pulse oximeter sensor communicatively; and a user interface configured to display physiological data received from the auscultation sensor and the pulse oximeter sensor and image data received from the camera and the multi-functional viewing tool.

### CROSS-REFERENCE TO RELATED APPLICATION

The present patent application claims the benefit of U.S. Provisional Patent Application No. 63/055,681 filed on Jul. 23, 2021, the entire contents of which are incorporated herein by reference.

### SUMMARY

Healthcare is expanding with the improvements in telemedical devices that can be used in a patient's home, by the patient, to collect physiological data. These devices also provide, for example, access to individuals who require medical care but may not be able to travel to see medical professionals, such as clinicians, doctors, and physicians. However, current telemedical devices meant for personal use lack both data integration and data storage as well as the capability of providing collected data to medical professionals. Additionally, privacy concerns arise when data is shared across a network.

Accordingly, implementations of the present disclosure are generally directed to a multi-functional telemedical device that is configured to be used by a patient to collect physiological data and provide the collected data to a medical professional (e.g., via a network). In one aspect, the described telemedical device includes diagnostic tools that a patient can use to measure their own physiological data. This collected data can be employed to improve medical diagnosis.

In one aspect, disclosed herein, are multi-functional telemedical devices comprising a housing configured for hand-held manipulation having an end that is configured to be fully rotational about an axis; a camera disposed on a front side of the end and communicatively; multi-functional viewing tool disposed a second side of the rotatable end and opposite the camera; an auscultation sensor communicatively; a pulse oximeter sensor communicatively; and a user interface configured to display physiological data received from the auscultation sensor and the pulse oximeter sensor and image data received from the camera and the multi-functional viewing tool. In some embodiments, the axis is located through a lengthwise midpoint of the housing and extending into a base of the at least one end. In some embodiments, the user interface comprises a tactile interface or a touch screen. In some embodiments, the multi-functional viewing tool comprises a light source and a magnifying lens. In some embodiments, the multi-functional viewing tool comprises an otoscopic attachment, a laryngoscopic attachment, or both. In some embodiments, the telemedical devices further comprise a charging stand. In some embodiments, the charging stand comprises a storage unit. In some embodiments, the otoscopic attachment and the laryngoscopic attachment are detachable and disposable. In some embodiments, the storage unit is configured to house the otoscopic attachment, a laryngoscopic attachment. In some embodiments, the telemedical devices further comprise a sphygmomanometer; and a heart rate sensor. In some embodiments, the telemedical devices further comprise an infrared light source; and a temperature measuring sensor configured to detect infrared light. In some embodiments, the pulse oximeter sensor is configured to detect infrared light. In some embodiments, the transceiver is configured to communicate via Bluetooth or Wi-Fi. In some embodiments, the physiological data comprises body temperature, blood pressure, pulse rate, respiratory rate, height, or weight. In some embodiments, the telemedical devices further comprise a light emitting diode (LED); a black light source; or an ultraviolet light source.

In another aspect, disclosed herein, are multi-functional telemedical devices comprising a processor; a housing configured for hand-held manipulation having an end that is configured to be fully rotational about an axis; a camera disposed on a front side of the end and communicatively coupled to the processor; an auscultation sensor communicatively coupled to the processor; a pulse oximeter sensor communicatively coupled to the processor; a user interface; and a computer-readable storage media coupled to the processor and having instructions stored thereon which, when executed by the processor, cause the processor to perform operations comprising: receiving physiological data from the auscultation sensor or the pulse oximeter sensor, or image data from the camera; determining display data based on the received physiological data or image data; and providing the display data to the user interface. In some embodiments, the user interface is configured to display the display data. In some embodiments, the telemedical devices further comprise a multi-functional viewing tool communicatively coupled to the processor and disposed a second side of the rotatable end and opposite the camera. In some embodiments, the multi-functional viewing tool comprises a light source and a magnifying lens. In some embodiments, the multi-functional viewing tool comprises an otoscopic attachment, a laryngoscopic attachment, or both. In some embodiments, the telemedical devices further comprise a transceiver configured to communicate with a network. In some embodiments, the operations further comprise: providing the received data to a back-end service or a user device via the network. In some embodiments, the axis is located through a lengthwise midpoint of the housing and extending into a base of the at least one end. In some embodiments, the user interface comprises a tactile interface or a touch screen. In some embodiments, the telemedical devices further comprise a charging stand. In some embodiments, the charging stand comprises a storage unit. In some embodiments, the otoscopic attachment and the laryngoscopic attachment are detachable and disposable. In some embodiments, the storage unit is configured to house the otoscopic attachment, a laryngoscopic attachment. In some embodiments, the telemedical devices further comprise a sphygmomanometer; and a heart rate sensor. In some embodiments, the telemedical devices further comprise an infrared light source; and a temperature measuring sensor configured to detect infrared light. In some embodiments, the pulse oximeter sensor is configured to detect infrared light. In some embodiments, the transceiver is configured to communicate via Bluetooth or Wi-Fi. In some embodiments, the physiological data comprises body temperature, blood pressure, pulse rate, respiratory rate, height, or weight. In some embodiments, the telemedical devices further comprise an LED; a black light source; or an ultraviolet light source.

In another aspect, disclosed herein, are multi-functional telemedical devices comprising a housing configured for hand-held manipulation having at least one end that is configured to be fully rotational about an axis; a camera disposed on a front side of the rotatable end; an infrared light source; an auscultation sensor; a pulse oximeter sensor configured to read infrared light; a multi-functional viewing tool comprising a light source, a magnifying lens, an otoscopic attachment and a laryngoscopic attachment, wherein the multi-functional viewing tool is disposed a second side of the rotatable end and opposite the camera; a sphygmomanometer; a heart rate sensor; a temperature senor configured to read infrared light and a user interface configured to display physiological data received from the auscultation sensor, the pulse oximeter sensor, the sphygmomanometer, the heart rate sensor, and the temperature measuring sensor and image data received from the camera and the multi-functional viewing tool. In some embodiments, the telemedical devices further comprise a charging stand. In some embodiments, the charging stand comprises a storage unit, wherein the otoscopic attachment and the laryngoscopic attachment are detachable and disposable. In some embodiments, the storage unit is configured to house the otoscopic attachment, a laryngoscopic attachment. In some embodiments, the telemedical devices further comprise a user interface configured to display the display data determined based on physiological data received from one of the sensors or image data received from the camera. In some embodiments, the axis is located through a lengthwise midpoint of the housing and extending into a base of the at least one end. In some embodiments, the user interface comprises a tactile interface or a touch screen. In some embodiments, the telemedical devices further comprise a transceiver configured to communicate with a network. In some embodiments, the pulse oximeter sensor is configured to detect infrared light. In some embodiments, the transceiver is configured to communicate via Bluetooth or Wi-Fi. In some embodiments, the physiological data comprises body temperature, blood pressure, pulse rate, respiratory rate, height, or weight. In some embodiments, the telemedical devices further comprise an LED; a black light source; or an ultraviolet light source. Particular implementations of the subject matter described in this disclosure can be implemented so as to realize one or more, but not limited to, of the following advantages. In some embodiments, the described telemedical device can be employed to measure physiological data (e.g., vital signs), such as body temperature, blood pressure, pulse rate, respiratory rate, height, and weight as well as document image data. In some embodiments, the described telemedical device provides the collected data to a user interface. In some embodiments, the described telemedical device provides the collected data to a back-end service or directly to a computing device that is accessible by a medical professional. In some embodiments, the described telemedical device provides for communication between a medical professional and a user. In some embodiments, the described telemedical device includes a camera that is disposed on an end that is rotational about an axis. In some embodiments, the described telemedical device includes sensors of some form disposed on the opposite side of the rotating end.

It is appreciated that methods in accordance with the present disclosure can include any combination of the aspects and features described herein. That is, methods in accordance with the present disclosure are not limited to the combinations of aspects and features specifically described herein, but also may include any combination of the aspects and features provided.

The details of one or more implementations of the present disclosure are set forth in the accompanying drawings and the description below. Other features and advantages of the present disclosure will be apparent from the description and drawings, and from the claims.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1 depicts a block diagram of a non-limiting, exemplary embodiment of a telemedical device;

FIGS. 2A-2D depict various views of a non-limiting, exemplary embodiment of the telemedical device;

FIGS. 3A-3C depict various views of another non-limiting, embodiment of the telemedical device;

FIGS. 4A-4C depict a non-limiting, embodiment of the telemedical device positioned on a charging stand;

FIG. 5 depicts a flow diagram of a non-limiting, example process for monitoring a user via a multi-functional telemedical device;

FIG. 6 depicts a block diagram of a non-limiting, exemplary computer system that can be programmed or otherwise configured to implement methods or systems of the present disclosure; and

FIG. 7 depicts a non-limiting, example environment that can be employed to execute implementations of the present disclosure.

### DETAILED DESCRIPTION

Before any embodiments of the disclosure are explained in detail, it is to be understood that the disclosure is not limited in its application to the details of embodiment and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising” or “having” and variations thereof herein is meant to encompass the items listed thereafter and equivalents thereof as well as additional items. The terms “mounted,” “connected” and “coupled” are used broadly and encompass both direct and indirect mounting, connecting, and coupling. Further, “connected” and “coupled” are not restricted to physical or mechanical connections or couplings, and can include electrical or hydraulic connections or couplings, whether direct or indirect.

It should also be noted that a plurality of hardware and software-based devices, as well as a plurality of different structural components may be used to implement the implementations. In addition, implementations may include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one implementation, the electronic based aspects of the disclosure may be implemented in software (for example, stored on non-transitory computer-readable medium) executable by one or more processors. As such, it should be noted that a plurality of hardware and software-based devices, as well as a plurality of different structural components may be utilized to implement various implementations. It should also be understood that although certain drawings illustrate hardware and software located within particular devices, these depictions are for illustrative purposes only. In some implementations, the illustrated components may be combined or divided into separate software, firmware and/or hardware. For example, instead of being located within and performed by a single electronic processor, logic and processing may be distributed among multiple electronic processors. Regardless of how they are combined or divided, hardware and software components may be located on the same computing device or may be distributed among different computing devices connected by one or more networks or other suitable communication links.

Embodiments of the present disclosure are generally directed to telemedical devices for collecting physiological data. More particularly, embodiments of the present disclosure are directed to multi-functional telemedical devices configured to be used by a patient to collect physiological data and provide the collected data to a medical professional.

Accordingly, described herein, in certain embodiments, are multi-functional telemedical devices comprising a housing configured for hand-held manipulation having an end that is configured to be fully rotational about an axis; a camera disposed on a front side of the end and communicatively; multi-functional viewing tool disposed a second side of the rotatable end and opposite the camera; an auscultation sensor communicatively; a pulse oximeter sensor communicatively; and a user interface configured to display physiological data received from the auscultation sensor and the pulse oximeter sensor and image data received from the camera and the multi-functional viewing tool. In some embodiments, the axis is located through a lengthwise midpoint of the housing and extending into a base of the at least one end.

Also described herein, in certain embodiments, are multi-functional telemedical devices comprising a processor; a housing configured for hand-held manipulation having an end that is configured to be fully rotational about an axis; a camera disposed on a front side of the end and communicatively coupled to the processor; an auscultation sensor communicatively coupled to the processor; a pulse oximeter sensor communicatively coupled to the processor; a user interface; and a computer-readable storage media coupled to the processor and having instructions stored thereon which, when executed by the processor, cause the processor to perform operations comprising: receiving physiological data from the auscultation sensor or the pulse oximeter sensor, or image data from the camera; determining display data based on the received physiological data or image data; and providing the display data to the user interface.

Also described herein, in certain embodiments, are multi-functional telemedical devices comprising a housing configured for hand-held manipulation having at least one end that is configured to be fully rotational about an axis; a camera disposed on a front side of the rotatable end; an infrared light source; an auscultation sensor; a pulse oximeter sensor configured to read infrared light; a multi-functional viewing tool comprising a light source, a magnifying lens, an otoscopic attachment and a laryngoscopic attachment, wherein the multi-functional viewing tool is disposed a second side of the rotatable end and opposite the camera; a sphygmomanometer; a heart rate sensor; a temperature senor configured to read infrared light and a user interface configured to display physiological data received from the auscultation sensor, the pulse oximeter sensor, the sphygmomanometer, the heart rate sensor, and the temperature measuring sensor and image data received from the camera and the multi-functional viewing tool.

### Definitions

Unless otherwise defined, all technical terms used herein have the same meaning as commonly understood by one of ordinary skill in the art to which the present subject matter belongs. As used in this specification and the appended claims, the singular forms “a,” “an,” and “the” include plural references unless the context clearly dictates otherwise. Any reference to “or” herein is intended to encompass “and/or” unless otherwise stated.

As used herein, the term “telemedical device” refers to any electronic device that is operated by a user and receives physiological or image data that can be saved and shared. This includes any electronic device that allows the user to access health care services remotely.

As used herein, the term “auscultation sensor” refers to any kind of medical listening device, including a stethoscope or a microphone, to listen to the sounds made by the user's body.

As used herein, the term “temperature sensor” or “temperature measuring sensor” refers to any kind of device that measures temperature.

As used herein, the term “pulse oximeter sensor” or “pulse oximeter” refers to any electronic device used to actively measure blood oxygen saturation in the body.

As used herein, the term “tactile interface” refers to any input system or method of input from the user in which the user is physically pressing buttons in order to interact with the user interface.

As used herein, the term “viewing tool” refers to any tool, including a camera or an otoscope, that is used to observe the condition of the human body from the user's point of view.

As used herein, the term “otoscopic attachment” refers to any tool used to look into the ears of the user or visually analyze any portion of the ear region of the user.

As used herein, the term “laryngoscopic attachment” refers to any tool used to look into the throat of the user or visually analyze any portion of the throat and mouth region of the user.

As used herein, the term “sphygmomanometer” refers to any instrument used to measure blood pressure of the user, including blood pressure cuffs.

As used herein, the term “heart rate sensor” refers to any instrument used to measure the heart rate of the user, including electrocardiography.

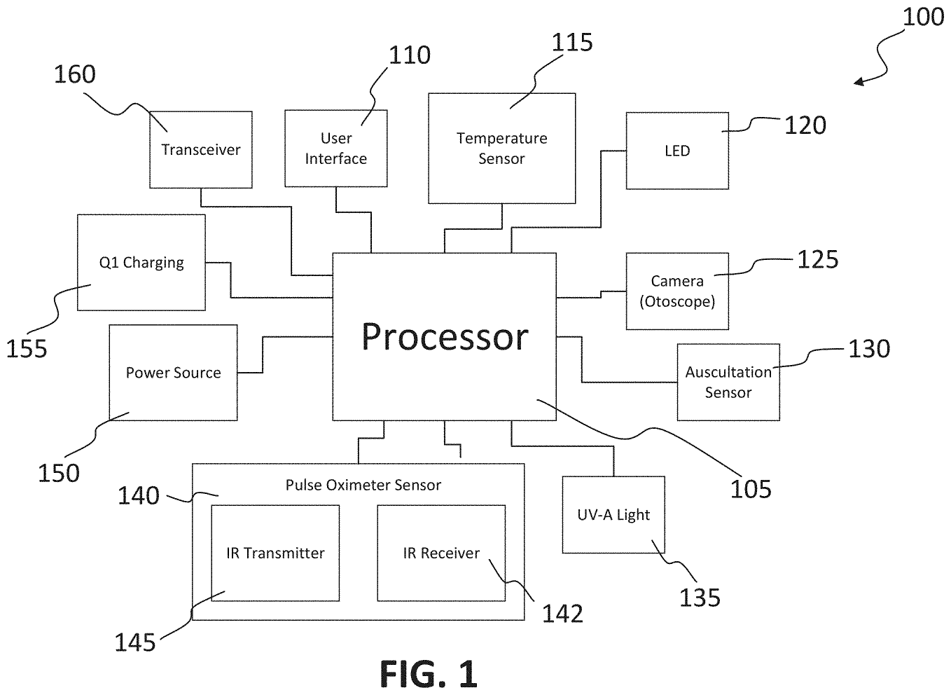

FIG. 1 depicts a block diagram of an exemplary embodiment of a telemedical device 100 based on the present disclosure. As depicted, the telemedical device 100 includes a processor 105, a user interface 110, a temperature measuring sensor (e.g., an infrared thermometer, or a thermopile) 115, an LED 120, a camera or otoscope 125, an auscultation sensor 130, an ultraviolet A (UV-A) light 135, a pulse oximeter sensor 140 that includes an infrared receiver 142 and an infrared transmitter 145, a power source (e.g., an ion battery) 150, Q1 or Qi charging receiver 155, and a transceiver 160.

In some embodiments, the user interface 110 comprises one or more input/output devices (e.g., display screen, a touch screen, a touch pad, a tactile interface, or the like). In some embodiments, the input/output device allows a user to interact with the device 100 by receiving instructions (e.g., via a touch screen, touch pad, tactile interface, or other input device) and providing information (e.g., via a display screen).

In some embodiments, the processor 105 processes data received from the various components of the telemedical device 100. In some embodiments, the processor 105 is communicatively coupled to each of the components of the telemedical device 100. In some embodiments, the processor 105 is configured to receive instructions from the user interface 110 and provide control information to the various device components. For example, in some embodiments, the processor 105 is configured to receive an instruction from the user interface 110 to activate one of the components and provide control instructions to the component to activate (e.g., turn on). In some embodiment, the processor 105 is a printed circuit board (PCB) that includes one or more communicatively coupled processors.

In some embodiments, the temperature measuring sensor 115 is configured to measure body temperature by translating the heat transfer emitted from a user to a voltage output. For example, in some embodiments, the temperature measuring sensor 115 can be held in proximity to a user's skin in order to measure the user's body temperature. In some embodiments, the temperature measuring sensor 115 provides the voltage output to the processor 105, which is configured to associate the voltage with a temperature value according to the specific temperature measuring sensor 115. In some embodiments, the processor 105 is configured to provide the temperature value to the user interface 110 for display.

In some embodiments, the LED 120 is configured to provide a light source to, for example, illuminate aspects of the environment while the device 100 is in use.

In some embodiments, the camera or otoscope 125 is configured to collect image data. In some embodiments, the camera or otoscope 125 is configured to provide collected image data to the processor 105. In some embodiments, the processor 105 is configured to provide the image data (e.g., as an image) to the user interface 110. For example, in some embodiments, the camera or otoscope 125 can be employed to view portions of a user's body (e.g., ears, nose, throat). In some embodiments, the camera or otoscope 125 includes a plurality of attachments to assist in capturing relevant data and images of specific portions of the user's body. In some embodiments, the attachments are coupled to the housing via snap fit, a screw-like configuration, and the like.

In some embodiments, the auscultation sensor 130 is configured to collect sound data. For example, in some embodiments, the auscultation sensor 130 (or a housing for the microphone) is configured to be applied to a user's skin near the relevant anatomical location to detect sounds inside the user's body such as sounds generated by, for example, the user's lungs or heart. In some embodiments, the auscultation sensor 130 is configured to provide collected sound data to the processor 105. In some embodiments, the processor 105 is configured to provide the sound data (e.g., as a sound file) to the user interface 110. For example, in some embodiments, a user can employ the microphone or stethoscope 130 to collect sound and listen to the sounds emitted from their heart or lungs.

In some embodiments, the UV-A light 135 is configured to emit ultraviolet light. In some embodiments, the UV-A light 135 can be employed in the treatment of various skin conditions. For example, in some embodiments, ultraviolet emitted from the UV-A light 135 is applied to the skin (e.g., with the UV-A light 135 in proximity to the skin) periodically. In some embodiments, ultraviolet emitted from the UV-A light 135 is applied for 1 minute, 5 minutes, 10 minutes, 15 minutes, 20 minutes, or 30 minutes.

In some embodiments, the pulse oximeter sensor 110 includes a port for receiving a user's figure.

In some embodiments, the infrared transmitter 145 is configured to produce infrared light, which, for example, can be applied to a translucent portion of a user's skin. When applied in this manner, a portion of the light is absorbed, and a portion is reflected. The portion of reflected light can be measured to determine an amount of hemoglobin in the user's blood. As such, in some embodiments, the infrared receiver 142 is configured to receive the reflected light and provide this data to the processor 105. In some embodiments, the processor 105 is configured to determine blood oxygen saturation based on this received data. In some embodiments, the processor 105 is configured to provide the pulse oximeter sensor 140 information to the user interface 110 for display. In some embodiments, the infrared receiver 142 and the infrared transmitter 145 form a single transceiver component.

In some embodiments, the ion battery 150 is rechargeable and provides power to the device 100. In some embodiments, the ion battery 150 employs the Q1 or Qi wireless charging receiver 155 for charging.

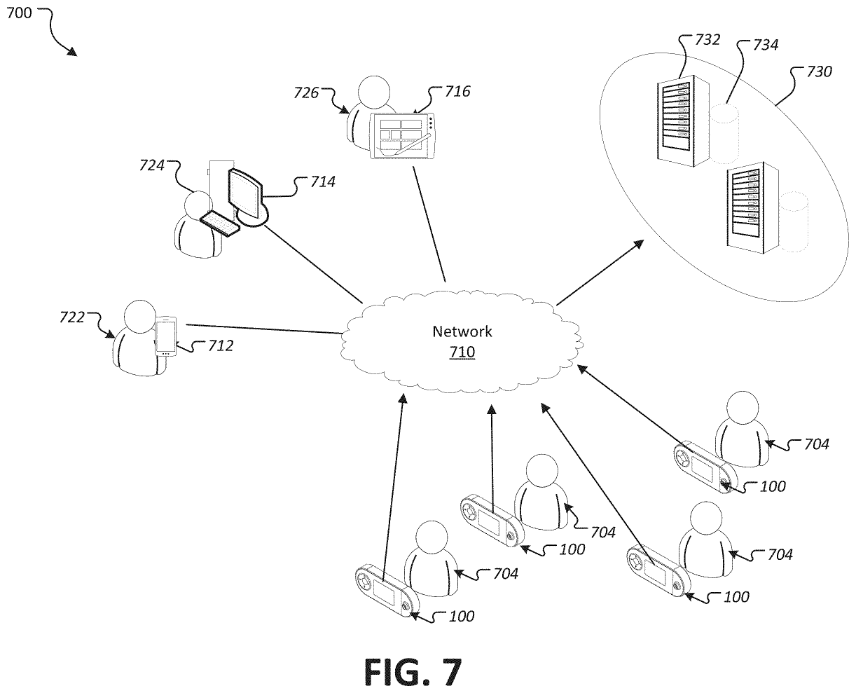

In some embodiments, the transceiver 160 is configured to send and receive information via, for example, a network. In some embodiments, as depicted in FIG. 7, the device 100 is configured to communicate, via the transceiver 160, with other devices and services such as a backend service or a user device.

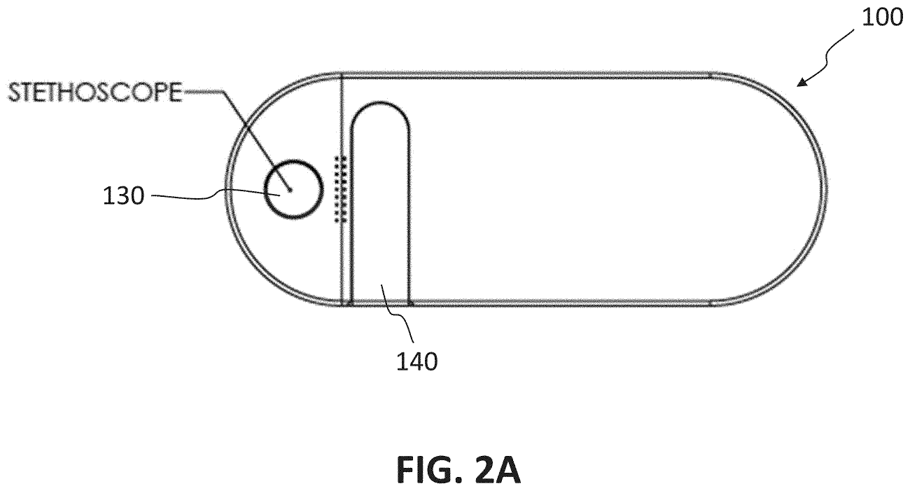

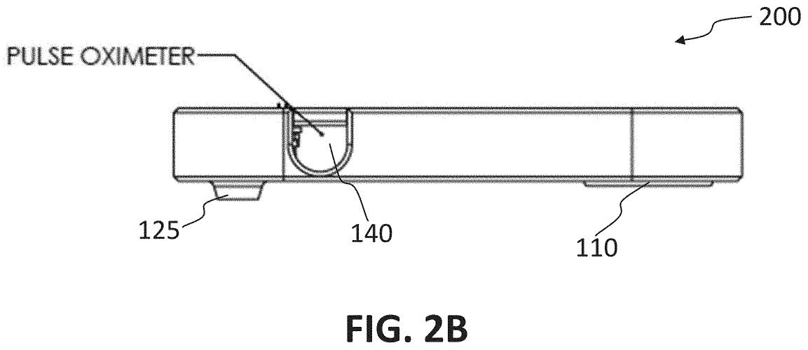

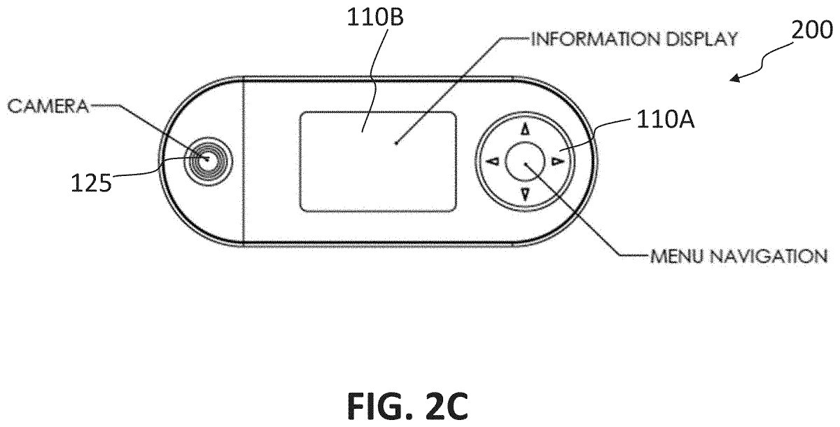

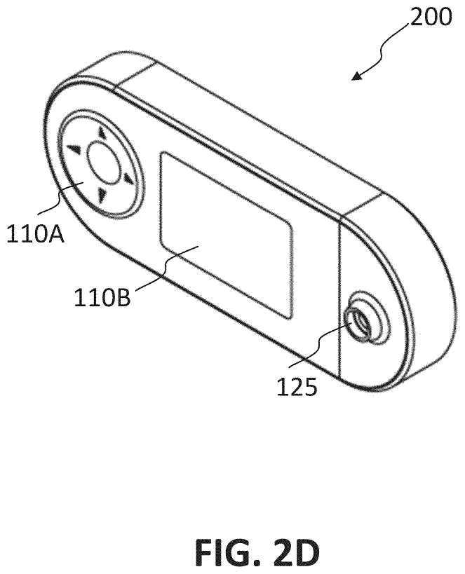

FIGS. 2A-2D depict various views of an exemplary embodiment of the telemedical device 100. As depicted, the telemedical device includes both a tactile interface 110A and a display screen 110B as the user interface 110. Other embodiments, include, for example, a user interface that is a touch screen.

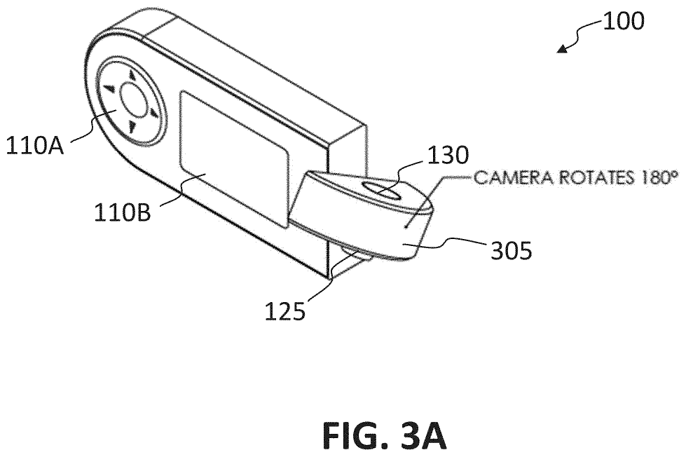

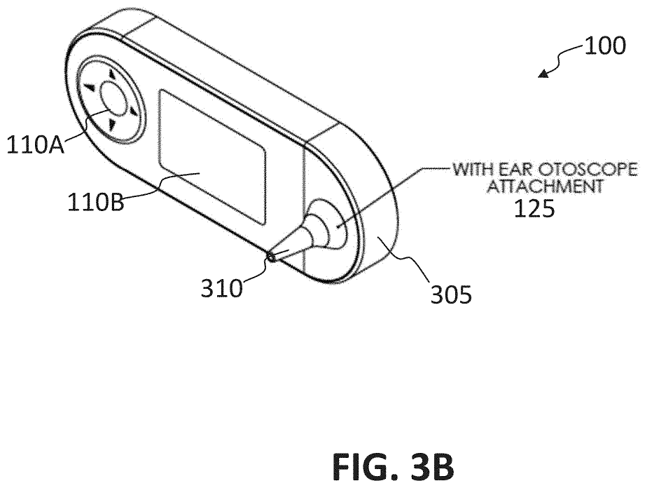

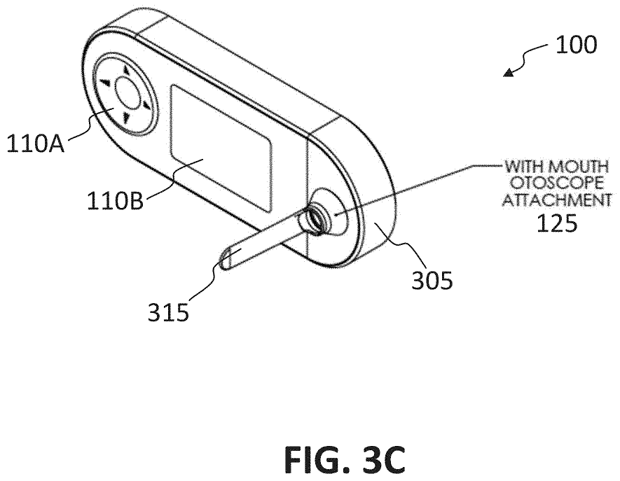

FIGS. 3A-3C depict various views of an exemplary embodiment of the telemedical device 100. As depicted, the device 100 includes a first end 305 and a second end where the tactile interface 110A is located. The end 305 of the telemedical device 100 is configured to rotate about an axis. In some embodiments, the end 305 is configured to rotate 360 degrees. As depicted, the camera or otoscope 125 and the auscultation sensor 130 are disposed on opposite sides of the rotatable end 305. In some embodiments, each side of the rotatable end 305 includes a camera or otoscope 125.

FIG. 3B depicts an exemplary embodiment of the telemedical device 100 that includes an ear attachment 310 for the otoscope 125. In some embodiments, the ear attachment 310 is configured to focus light into a user's ear.

FIG. 3C depicts an exemplary embodiment of the telemedical device 100 that includes a mouth attachment 315 (e.g., a spatula or tongue depressor) for the otoscope 125. In some embodiments, the mouth attachment 315 is configured to focus light into a user's mouth and throat.

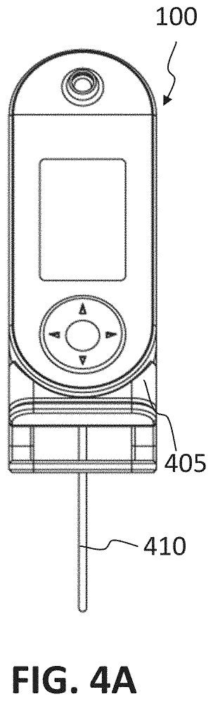

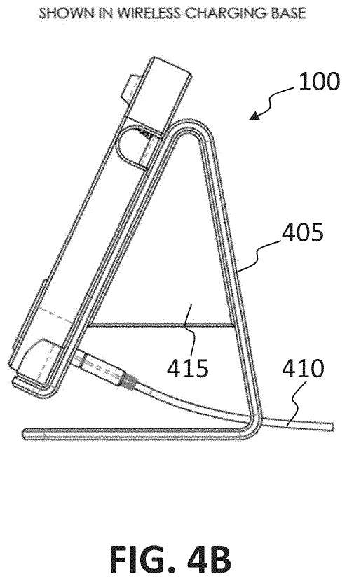

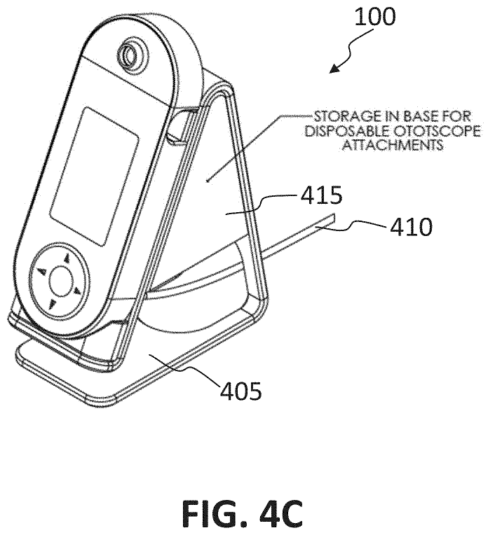

FIGS. 4A-4C depict an exemplary embodiment of the telemedical device 100 positioned on a charging stand 405 and attached to a charging cord 410. In some embodiments, the charging stand 405 is configured for Q1 or Qi wireless charging via the Q1 or Qi charging receiver 155.

FIG. 4C depicts an exemplary embodiment where the charging stand 405 includes a storage unit 415. In some embodiments, the storage unit 415 houses ear attachments 310 and mouth attachment 315 for the otoscope 125, as depicted in FIGS. 3A-3C.

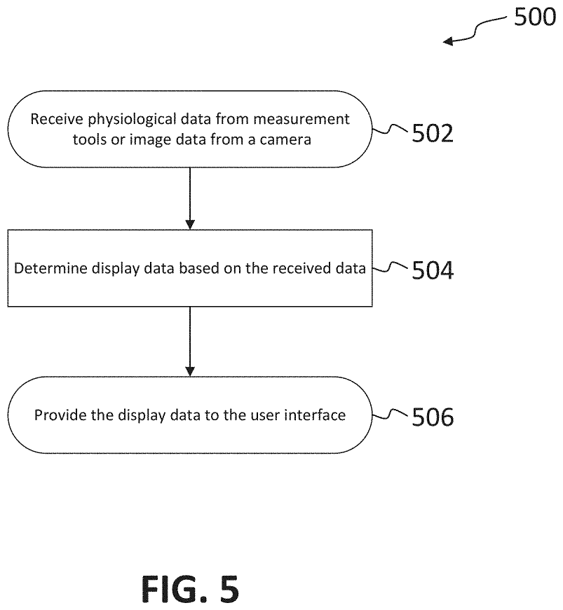

FIG. 5 depicts a flow diagram of an example process 500 for monitoring a user via a multi-functional telemedical device. In some embodiments, the multi-functional medical device includes a processor configured to execute the process 500. For clarity of presentation, the description that follows generally describes the process 500 in the context of FIGS. 1-4C and 6-7. However, it will be understood that the process 500 may be performed, for example, by any other suitable system or a combination of systems as appropriate.

In some embodiments, the multi-functional telemedical device includes a housing configured for hand-held manipulation and having at least one end that is configured to be fully rotational about an axis. In some embodiments, the multi-functional telemedical device includes a camera that is coupled to the processor and disposed on a front side of the rotatable end. In some embodiments, the multi-functional telemedical device includes a multi-functional viewing tool that is coupled to the processors and disposed a second side of the rotatable end and opposite the camera. In some embodiments, the multi-functional telemedical device includes a second camera that is communicatively coupled to the processors and disposed on the second side of the rotatable end opposite the first camera. In some embodiments, the axis is located through the lengthwise midpoint of the housing and extends into the base of the rotatable end. In some embodiments, the multi-functional telemedical device includes an auscultation sensor that is communicatively coupled to the processors. In some embodiments, the multi-functional telemedical device includes a pulse oximeter sensor that is communicatively coupled to the processors.

In some embodiments, the multi-functional telemedical device includes a user interface that is communicatively coupled to the processors. In some embodiments, the user interface comprises a tactile interface or a touch screen. In some embodiments, the user interface comprises a display screen. In some embodiments, the user interface comprises a touch screen.

In some embodiments, the multi-functional viewing tool includes a magnifying lens. In some embodiments, the multi-functional viewing tool includes an otoscopic attachment, a laryngoscopic attachment, or both. In some embodiments, the attachments are detachable and disposable.

In some embodiments, the multi-functional telemedical device includes a sphygmomanometer that is communicatively coupled to the processors. In some embodiments, the multi-functional telemedical device includes a heart rate sensor that is communicatively coupled to the processors. In some embodiments, the multi-functional telemedical device includes a temperature measuring sensor that is communicatively coupled to the processors.

In some embodiments, the multi-functional telemedical device includes a charging stand. In some embodiments, the charging stand includes a storage unit. In some embodiments, the storage unit is configured to house the attachments.

In some embodiments, the at least one light source comprises an LED. In some embodiments, the at least one light source comprises a black light source. In some embodiments, the at least one light source comprises an ultraviolet light source. In some embodiments, the at least one light source comprises an infrared light source. In some embodiments, the pulse oximeter sensor and the temperature measuring sensor are configured to detect infrared light.

In some embodiments, the multi-functional telemedical device includes a transceiver that is configured to communicate with a network. In some embodiments, the transceiver is configured to communicate via Bluetooth or Wi-Fi.

At 502, physiological data or image data is received from the auscultation sensor, the pulse oximeter sensor, or one of the cameras. In some embodiments, the physiological data includes body temperature, blood pressure, pulse rate, respiratory rate, height, or weight. From 502, the process 500 proceeds to 504.

At 504, display data is determined based on the received physiological data or image data. In some embodiments, the transceiver is configured to provide the received data to a back-end service or a user device via the network. From 504, the process 500 proceeds to 506.

At 506, the display data is provided to the user interface. In some embodiments, the user interface is configured to display the display data. From 506, the process 500 ends.

### Processing Devices and Processors

In some embodiments, the platforms, systems, media, and methods described herein include a computer, or use of the same. In further embodiments, the computer includes one or more hardware central processing units (CPUs) or general-purpose graphics processing units (GPGPUs) that carry out the device's functions. In still further embodiments, the computer comprises an operating system configured to perform executable instructions. In some embodiments, the computer is optionally connected a computer network. In further embodiments, the computer is optionally connected to the Internet such that it accesses the World Wide Web. In still further embodiments, the computer is optionally connected to a cloud computing infrastructure. In other embodiments, the computer is optionally connected to an intranet. In other embodiments, the computer is optionally connected to a data storage device.

In accordance with the description herein, suitable computers include, by way of non-limiting examples, server computers, desktop computers, laptop computers, notebook computers, sub-notebook computers, netbook computers, netpad computers, handheld computers, Internet appliances, mobile smartphones, tablet computers, vehicles as well as the described multi-functional telemedical device. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the computer includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google Android®, Microsoft® Windows Phone® OS, Microsoft® Windows Mobile® OS, Linux, and Palm® WebOS®.

In some embodiments, the device includes a storage or memory device. The storage or memory device is one or more physical apparatuses used to store data or programs on a temporary or permanent basis. In some embodiments, the device is volatile memory and requires power to maintain stored information. In some embodiments, the device is non-volatile memory and retains stored information when the computer is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, compact disc read-only memories (CD-ROMs), digital versatile discs (DVDs), flash memory devices, magnetic disk drives, magnetic tapes drives, optical disk drives, and cloud computing-based storage. In further embodiments, the storage or memory device is a combination of devices such as those disclosed herein.

In some embodiments, the computer includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the computer, such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In some embodiments, the computer includes an input device to receive information from a user. In some embodiments, the input device is a keyboard. In some embodiments, the input device is a pointing device including, by way of non-limiting examples, a mouse, trackball, track pad, joystick, tactile interface, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

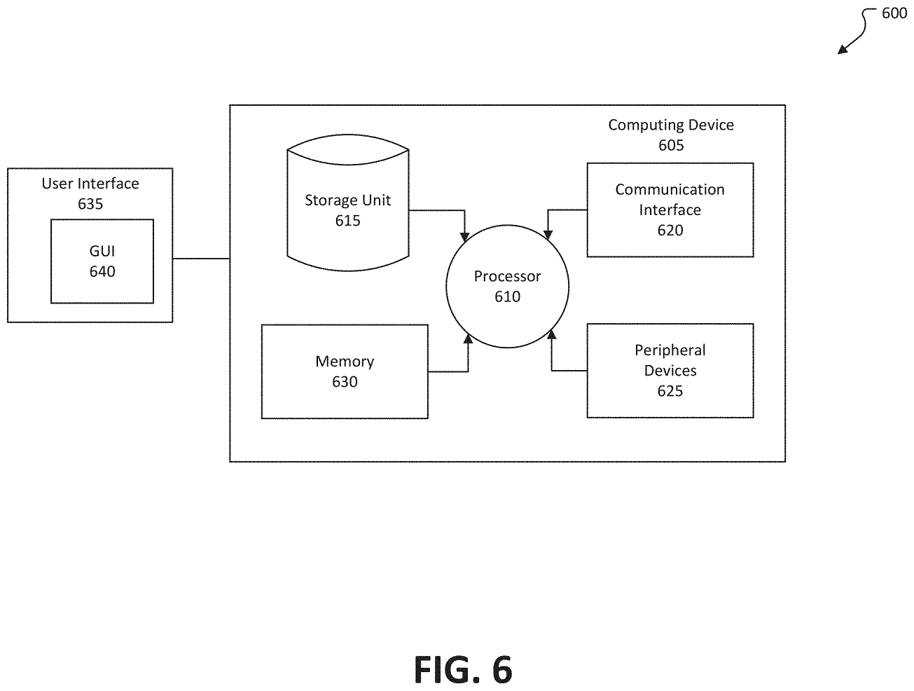

Computer systems are provided herein that can be used to implement methods or systems of the disclosure. FIG. 6 depicts an example 600 of a computer system 605 that can be programmed or otherwise configured to implement methods or systems of the present disclosure. For example, the computing device 605 can be programmed or otherwise configured to provide calculated results or values based on physiological or image data received via the various input devices (e.g., such as described above as included within the described multi-functional telemedical device 100).

In the depicted embodiment, the computing device 605 includes a CPU (also “processor” and “computer processor” herein) 610, which is optionally a single core, a multi core processor, or a plurality of processors for parallel processing. The computing device 605 also includes memory or memory location 630 (e.g., random-access memory, read-only memory, flash memory), electronic storage unit 615 (e.g., hard disk), communication interface 620 (e.g., network adapter, or transceiver) for communicating with one or more other systems, and peripheral devices 625, such as cache, other memory, data storage, electronic display adapters, cameras, or measurement tools. The memory 630, storage unit 615, interface 620, and peripheral devices 625 are in communication with the CPU 610 through a communication bus (solid lines), such as a motherboard or a PCB. The storage unit 615 comprises a data storage unit (or data repository) for storing data. The computing device 605 is optionally operatively coupled to a computer network, such as the network 710 depicted in FIG. 7, with the aid of the communication interface 620.

In some embodiments, the CPU 610 can execute a sequence of machine-readable instructions, which can be embodied in a program or software. The instructions may be stored in a memory location, such as the memory 630. The instructions can be directed to the CPU 610, which can subsequently program or otherwise configure the CPU 610 to implement methods of the present disclosure. Examples of operations performed by the CPU 610 can include fetch, decode, execute, and write back. In some embodiments, the CPU 610 is part of a circuit, such as an integrated circuit. One or more other components of the computing device 605 can be optionally included in the circuit. In some embodiments, the circuit is an application specific integrated circuit (ASIC) or a FPGA.

In some embodiments, the storage unit 615 stores files, such as drivers, libraries and saved programs. In some embodiments, the storage unit 615 stores user data, e.g., user preferences and user programs. In some embodiments, the computing device 605 includes one or more additional data storage units that are external, such as located on a remote server that is in communication through an intranet or the internet.

In some embodiments, the computing device 605 communicates with one or more remote computer systems through a network. For instance, the computing device 605 can communicate with a remote computer system. Examples of remote computer systems include personal computers (e.g., portable PC), slate or tablet PCs (e.g., Apple® iPad, Samsung® Galaxy Tab, etc.), smartphones (e.g., Apple® iPhone, Android-enabled device, Blackberry®, etc.), or personal digital assistants. In some embodiments, a user can access the computing device 605 via a network.

In some embodiments, methods as described herein are implemented by way of machine (e.g., computer processor) executable code stored on an electronic storage location of the computing device 605, such as, for example, on the memory 630 or the electronic storage unit 615. In some embodiments, the CPU 610 is adapted to execute the code. In some embodiments, the machine executable or machine-readable code is provided in the form of software. In some embodiments, during use, the code is executed by the CPU 610. In some embodiments, the code is retrieved from the storage unit 615 and stored on the memory 630 for ready access by the CPU 610. In some situations, the electronic storage unit 615 is precluded, and machine-executable instructions are stored on the memory 630. In some embodiments, the code is pre-compiled. In some embodiments, the code is compiled during runtime. The code can be supplied in a programming language that can be selected to enable the code to execute in a pre-compiled or as-compiled fashion.

In some embodiments, the computing device 605 can include or be in communication with a user interface 635 (e.g., user interface 110). In some embodiments, the user interface 635 provides a graphical user interface (GUI) 640.

FIG. 7 depicts an example environment 700 where the telemedical devices 100 can be employed to, for example, provide physiological data and image data collected from the users (e.g., patients) 704 to medical professionals 724 (e.g., caretakers, doctors, physician assistants, nurses, and the like). As depicted, the example environment 700 includes telemedical devices 100, computing devices 712, 714, 716, a back-end system 730, and a network 710.

In some embodiments, the example environment 700 is deployed (at least partially) within a hospital or health care center. In some embodiments, the telemedical devices 100 provide physiological data and image data to the computing devices 712, 714, 716, which are accessible by the medical professionals 722, 724, 726. In some embodiments, the medical professionals 722, 724, 726 provide the telemedical devices 100 to the users 704. In some embodiments, the users 704 are in various rooms or otherwise dispersed through the facility to which the example environment 700 is deployed. In some embodiments, the users 704 are at the home or other private setting. In some embodiments, the users 704 can employ the various sensors and sensors on their respective telemedical device 100 to collect the appropriate physiological data. In some embodiments, the users 704 can employ the camera to collect a measurement of their height. In some embodiments, the users 704 can enter their weight or height via the user interface 110 provided by their respective telemedical device 100. In some embodiment, the telemedical devices 100 is communicably coupled to a scale (e.g., via the network 710 or directly via Bluetooth) and the user's 704 weight is collected from the scale.

In some embodiments, the network 710 includes a local area network (LAN), a wide area network (WAN), the Internet, or a combination thereof, and connects web sites, devices, and systems (e.g., the telemedical devices 100, the computing devices 712, 714, and 716, and the back-end system 730). In some embodiments, the network 710 includes the Internet, an internet, or an extranet. In some embodiments, the network 710 includes a telecommunication network or data network. In some embodiments, the network 710 can be accessed over a wired or a wireless communications link, such as a network gateway. For example, in some embodiments, a Bluetooth module or other type of transceiver in each of the telemedical devices 100 transmits a signal that is received by a network gateway (not shown) or the computing devices 712, 714, and 716. In some embodiments, the computing devices 712, 714, and 716 or telemedical devices 100 use a cellular network to access the network 710. In some embodiments, the network 710 includes a network of physical objects (or Internet of things) with mesh and start topological structures (e.g., Narrowband IoT (NBIOT), Long Range (LoRa, ZigBee, general package radio service ((GPRS), and Long-Term Evolution (LTE) category M1 (Cat1)). In some embodiments, protocols can be adopted within the the network 710 for specific applications and environments.

In the depicted example environment 700, the back-end system 730 includes at least one server device 732 and at least one data store 734. In some embodiments, the device 732 is sustainably similar to computing device 605 depicted in FIG. 6. In some embodiments, the server device 732 is a server-class hardware type device. In some embodiments, back-end system 730 includes computer systems using clustered computers and components to act as a single pool of seamless resources when accessed through the network 710. For example, such implementations may be used in data center, cloud computing, storage area network (SAN), and network attached storage (NAS) applications. In some embodiments, back-end system 730 is deployed using a virtual machine(s).

In some embodiments, the data store 734 is a repository for persistently storing and managing collections of data. In some embodiments, the data store 734 is a data repository that includes, for example, a database. In some embodiments, the data store 734 is a simpler data store and includes files, emails, and the like. In some embodiments, the data store 734 includes a database, which includes a series of bytes or an organized collection of data that is managed by a database management system (DBMS). In some embodiments, the data store 734 includes a distributed ledger (e.g., a blockchain).

In some embodiments, the computing devices 712, 714, and 716 are sustainably similar to computing device 605 depicted in FIG. 6. In some embodiments, the computing devices 712, 714, and 716 each include any appropriate type of computing device including a desktop computer, a laptop computer, a handheld computer, a tablet computer, a personal digital assistant (PDA), a cellular telephone, a network appliance, a camera, a smart phone, an enhanced general packet radio service (EGPRS) mobile phone, a media player, a navigation device, an email device, a game console, or an appropriate combination of any two or more of these devices or other data processing devices. In the depicted example, the computing device 712 is a smartphone, the computing device 714 is a tablet-computing device, and the computing device 716 is a desktop computing device. Three user computing devices 712, 714, and 716 and four telemedical devices 100 are depicted in FIG. 7 for simplicity. It is contemplated, however, that implementations of the present disclosure can be realized with any of the appropriate computing devices or telemedical devices, such as those mentioned previously. Moreover, implementations of the present disclosure can employ any number of devices as required.

In some embodiments, the at least one server system 732 hosts one or more computer-implemented services provided by the described telemedical device system 700 that users 722, 724, and 726 can interact with using the respective computing devices 712, 714, and 716. In some examples, the users 722, 724, and 726 interact with the provided services and the telemedical devices 100 through a GUI or application that is installed and executing on their respective computing devices 712, 714, and 716. For example, the users 704 may employ their respective telemedical device 100 to collect physiological data, which is provided to the back-end system 730 or directly to the computing devices 712, 714, and 716 via the network 710. In some embodiments, the back-end system 730 stores the received data in the data store 734.

In some embodiments, the users 722, 724, and 726 have access to provided physiological data (e.g., via a service provider by the back-end system 730 or an application executing on the computing device). In some embodiments, the users 722, 724, and 726 can communicate with each of the users 702 via the user's respective telemedical device 100. For example, in some embodiments, the computing devices 712, 714, and 716 provide information to the telemedical devices 100, which is displayed on the respective user interface 110 or otherwise provided to the respective user 704. In some embodiments, the users 722, 724, and 726 determine medical advice, diagnosis information, prescription information, and the like, based on the physiological data received from the telemedical devices 100. In some embodiments, the users 722, 724, and 726 provide, via the respective computing devices 712, 714, and 716, medical advice, diagnosis information, prescription information, and the like, to the users 702 via the telemedical devices 100.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computer. In further embodiments, a computer readable storage medium is a tangible component of a computer. In still further embodiments, a computer readable storage medium is optionally removable from a computer. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the computer's CPU, written to perform a specified task. Computer readable instructions may be implemented as program modules, such as functions, objects, API, data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft .NET or Ruby on Rails (RoR). In some embodiments, a web application utilizes one or more database systems including, by way of non-limiting examples, relational, non-relational, object oriented, associative, and eXtensible Markup Language (XML) database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or XML. In some embodiments, a web application is written to some extent in a presentation definition language such as Cascading Style Sheets (CSS). In some embodiments, a web application is written to some extent in a client-side scripting language such as Asynchronous JavaScript and XML (AJAX), Flash® ActionScript, JavaScript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile computer. In some embodiments, the mobile application is provided to a mobile computer at the time it is manufactured. In other embodiments, the mobile application is provided to a mobile computer via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

In some embodiments, the platforms, systems, media, and methods disclosed herein include software, server, or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Data Stores

In some embodiments, the platforms, systems, media, and methods disclosed herein include one or more data stores. In view of the disclosure provided herein, those of skill in the art will recognize that data stores are repositories for persistently storing and managing collections of data. Types of data stores repositories include, for example, databases and simpler store types, or use of the same. Simpler store types include files, emails, and the like. In some embodiments, a database is a series of bytes that is managed by a DBMS. Many databases are suitable for receiving various types of data, such as weather, maritime, environmental, civil, governmental, or military data. In various embodiments, suitable databases include, by way of non-limiting examples, relational databases, non-relational databases, object-oriented databases, object databases, entity-relationship model databases, associative databases, and XML databases. Further non-limiting examples include SQL, PostgreSQL, MySQL, Oracle, DB2, and Sybase. In some embodiments, a database is internet-based. In some embodiments, a database is web-based. In some embodiments, a database is cloud computing based. In some embodiments, a database is based on one or more local computer storage devices.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or XML. In some embodiments, a web application is written to some extent in a presentation definition language such as Cascading Style Sheets (CSS). In some embodiments, a web application is written to some extent in a client-side scripting language such as Asynchronous JavaScript and XML (AJAX), Flash® ActionScript, JavaScript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile computer. In some embodiments, the mobile application is provided to a mobile computer at the time it is manufactured. In other embodiments, the mobile application is provided to a mobile computer via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non limiting examples. AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™ Visual Basic, and VB .NET, or combinations thereof. Compilation is often performed, at least in part, to create an executable program. In some embodiments, a computer program includes one or more executable complied applications.

While preferred embodiments of the present disclosure have been shown and described herein, it will be obvious to those skilled in the art that such embodiments are provided by way of example only. Numerous variations, changes, and substitutions will now occur to those skilled in the art without departing from the described system. It should be understood that various alternatives to the embodiments described herein may be employed in practicing the described system.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Moreover, the separation or integration of various system modules and components in the implementations described earlier should not be understood as requiring such separation or integration in all implementations, and it should be understood that the described components and systems can generally be integrated together in a single product or packaged into multiple products. Accordingly, the earlier description of example embodiments does not define or constrain this disclosure. Other changes, substitutions, and alterations are also possible without departing from the spirit and scope of this disclosure.

Thus, the disclosure provides, among other things, a multi-functional telemedical device configured to monitor the physiological well-being of the user. Various features and advantages of the disclosure are set forth in the following claims.

What is claimed is:

1. A multi-functional telemedical device comprising:
a housing configured for hand-held manipulation having an end that is configured to be fully rotational about an axis;
a camera disposed on a front side of the end and communicatively;
multi-functional viewing tool disposed a second side of the rotatable end and opposite the camera;
an auscultation sensor communicatively;
a pulse oximeter sensor communicatively; and
a user interface configured to display physiological data received from the auscultation sensor and the pulse oximeter sensor and image data received from the camera and the multi-functional viewing tool.

2. The telemedical device of claim 1, wherein the axis is located through a lengthwise midpoint of the housing and extending into a base of the at least one end.

3. The telemedical device of claim 1, wherein the user interface comprises a tactile interface or a touch screen.

4. The telemedical device of claim 1, wherein the multi-functional viewing tool comprises a light source and a magnifying lens.

5. The telemedical device of claim 4, wherein the multi-functional viewing tool comprises an otoscopic attachment, a laryngoscopic attachment, or both.

6. The telemedical device of claim 5, further comprising a charging stand, wherein the charging stand comprises a storage unit.

7. The telemedical device of claim 6, wherein the otoscopic attachment and the laryngoscopic attachment are detachable and disposable, and wherein the storage unit is configured to house the otoscopic attachment, a laryngoscopic attachment.

8. The telemedical device of claim 1, further comprising:
a sphygmomanometer; and
a heart rate sensor.

9. The telemedical device of claim 1, further comprising:
an infrared light source; and
a temperature measuring sensor configured to detect infrared light, wherein the pulse oximeter sensor is configured to detect infrared light.

12. The telemedical device of claim 10, wherein the transceiver is configured to communicate via Bluetooth or Wi-Fi.

13. The telemedical device of claim 1, wherein the physiological data comprises body temperature, blood pressure, pulse rate, respiratory rate, height, or weight.

14. The telemedical device of claim 1, further comprising:
a light emitting diode (LED);
a black light source; or
an ultraviolet light source.

15. A multi-functional telemedical device comprising:
a processor;
a housing configured for hand-held manipulation having an end that is configured to be fully rotational about an axis;
a camera disposed on a front side of the end and communicatively coupled to the processor;
an auscultation sensor communicatively coupled to the processor;
a pulse oximeter sensor communicatively coupled to the processor;
a user interface; and
a computer-readable storage media coupled to the processor and having instructions stored thereon which, when executed by the processor, cause the processor to perform operations comprising:
receiving physiological data from the auscultation sensor or the pulse oximeter sensor, or image data from the camera;
determining display data based on the received physiological data or image data; and
providing the display data to the user interface, wherein the user interface is configured to display the display data.

16. The telemedical device of claim 15, further comprising a multi-functional viewing tool communicatively coupled to the processor and disposed a second side of the rotatable end and opposite the camera.

17. The telemedical device of claim 15, further comprising a transceiver configured to communicate with a network.

18. The telemedical device of claim 17, wherein the operations further comprise:
providing the received data to a back-end service or a user device via the network.

19. A multi-functional telemedical device comprising:
a housing configured for hand-held manipulation having at east one end that is configured to be fully rotational about an axis;
a camera disposed on a front side of the rotatable end;
an infrared light source;
an auscultation sensor;
a pulse oximeter sensor configured to read infrared light;
a multi-functional viewing tool comprising a light source, a magnifying lens, an otoscopic attachment and a laryngoscopic attachment, wherein the multi-functional viewing tool is disposed a second side of the rotatable end and opposite the camera;
a sphygmomanometer;
a heart rate sensor;
a temperature measuring sensor configured to read infrared light and
a user interface configured to display physiological data received from the auscultation sensor, the pulse oximeter sensor, the sphygmomanometer, the heart rate sensor, and the temperature measuring sensor and image data received from the camera and the multi-functional viewing tool.

20. The telemedical device of claim 19, further comprising a charging stand, wherein the charging stand comprises a storage unit, wherein the otoscopic attachment and the laryngoscopic attachment are detachable and disposable, and wherein the storage unit is configured to house the otoscopic attachment, a laryngoscopic attachment.
